(19) United States
(12) Patent Application Publication
(10) Pub. No.: US 2010/0166651 A1
(43) Pub. Date: Jul. 1, 2010

(54) Radiotherapeutic High Specific Activity Tin-117m and Methods of Use

(76) Inventor: Marlene OLIVER, West Richland, WA (US)

Correspondence Address: Adler & Associates, 8011 Candle Lane, Houston, TX, 77071, US

(21) Appl. No.: 12/086,949

(22) PCT Filed: Dec. 19, 2006
(86) PCT No.: PCT/US2006/048355

§ 371 Date: Jun. 20, 2008

Related U.S. Application Data
(60) Provisional application No. 60/752,237, filed on Dec. 20, 2005.

(51) Int. Cl. A61K 51/08; A61K 51/00; A61K 51/04; A61K 51/02; A61K 51/12; A61P 19/08; A61P 35/00; A61P 29/00; A61K 51/10; A61P 7/04

(52) U.S. Cl. 424/1.17; 424/1.11; 424/1.65; 424/1.69; 424/1.29; 424/1.37; 424/1.49

(57) ABSTRACT

The present invention provides a radiotherapeutic composition comprising a high specific activity tin-117m source of radiotherapeutic atomic electrons and a delivery vehicle, e.g., a chelator or nanosphere or microsphere effective to contain tin-117m therein. The present invention also provides a targeting moiety linked to the delivery vehicle. Further provided are methods of treating a bone-associated pathophysiological condition or imaging the same using the high specific activity radiotherapeutic compositions.

### CROSS-REFERENCE TO RELATED APPLICATIONS

This U.S. national stage application is filed under 35 U.S.C. 363 and claims benefit of priority under 35 U.S.C. 365 of international application PCT/US2006/048355, filed Dec. 19, 2006, now abandoned, which claims benefit of priority under 35 U.S.C. 119(e) of provisional U.S. Ser. No. 60/752,237, filed Dec. 20, 2005, now abandoned.

### BACKGROUND OF THE INVENTION

1. Field of the Invention

The present invention relates generally to the fields of nuclear physics and nuclear medicine. More specifically, the present invention relates to delivery vehicles comprising high specific activity tin-117m for use in radiotherapeutic applications.

2. Description of the Related Art

Every year approximately 320,000 new cases of bone cancer are reported in the United States. Also, over ten million suffer from arthritis and a much smaller number from ankylosing spondylitis and hemophilia. Tin-117m(Sn IV) chelated to dietheylenetriamine pentaacetic acid (DTPA) has been used in clinical trials as a bone seeking pain reliever for metastatic bone cancers which are currently untreatable and fatal. The tin-117m complex does not sedate the patient, as do narcotic drugs, and provides selective radiation to the metastatic bone tumor while providing little to the bone marrow. Thus, it does not interfere with the bone marrow's ability to fight infection or with blood clotting.

Tin-117m emits short-range atomic electrons and a 159 keV gamma ray. The specific activity of the tin-117m used in these chelates has been no more than 20 Ci/g. While effective to palliate bone pain caused by metastatic bone cancers, the tin-117m complexes have not been useful to treat the metastatic bone cancer itself.

The isotopes strontium-89, samarium-153, rhenium-186, erbium-169 and yttrium-90 are used as potential radiotherapeutic agents. However, the beta and gamma emissions from some of these isotopes, e.g., strontium-89, are regarded as too energetic to optimize treatment. Similarly, the emissions from others, e.g., samarium-153, are not energetic enough.

The effectiveness of a radioisotope that emits short-range particles is improved if the specific activity of a radioisotope construct can be increased and a construct can be designed to specifically target a site of interest. However, specific activity is often limited by the available production methods for the isotope. Therefore, a recognized need exists in the art for medically useful radionuclides with high specific activities that are targetable and have little or no effect on healthy tissue or organs.

Thus, the prior art is still deficient in the lack of high specific activity tin-117m. Specifically, the prior art is deficient in the lack of high specific activity tin-117m as a source of atomic electrons useful as a radiotherapeutic for primary and metastatic bone neoplasms or for bone joint diseases or disorders having an inflammatory component. The present invention fulfills this long-standing need and desire in the art.

### SUMMARY OF THE INVENTION

The present invention is directed to radiotherapeutic composition. The radiotherapeutic composition comprises a high specific activity tin-117m source of radiotherapeutic atomic electrons and a delivery vehicle effective to contain tin-117m therein. The present invention is directed to a radiotherapeutic composition further comprising a targeting moiety linked to the delivery vehicle.

The present invention also is directed to a related radiotherapeutic composition. The radiotherapeutic composition comprises a high specific activity stannous-117m chloride and a nanosphere or microsphere encapsulating the stannous-117m chloride. This related radiotherapeutic composition also comprises a targeting moiety linked to the nanosphere or microsphere.

The present invention also is directed to another related radiotherapeutic composition. The radiotherapeutic composition comprises a high specific activity stannic-117m chloride and a bifunctional chelator of stannic-117m chloride. This related radiotherapeutic composition also comprises a targeting moiety linked to the bifunctional chelator.

The present invention is directed further to a method of treating a bone-associated pathological condition. The method comprises administering one of the radiotherapeutic compositions described herein to the subject. The radiotherapeutic atomic electrons emitted by the high specific activity tin-117m comprising the composition treats the bone-associated pathological condition upon delivery thereto.

Other and further aspects, features, benefits, and advantages of the present invention will be apparent from the following description of the presently preferred embodiments of the invention given for the purpose of disclosure.

### DETAILED DESCRIPTION OF THE INVENTION

As used herein, the term “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method described herein can be implemented with respect to any other method described herein.

As used herein, the term “or” in the claims is used to mean “and/or” unless explicitly indicated to refer to alternatives only or the alternatives are mutually exclusive, although the disclosure supports a definition that refers to only alternatives and “and/or.”

As used herein, the phrase “bone-associated pathological condition” refers to deleterious conditions involving bone or bone cells, such as neoplastic diseases or disorders and inflammatory responses, including those associated with autoimmune and genetic diseases or disorders involving bone or bone cells, for example, but not limited to arthritis and hemophilia.

As used herein, the term “neoplasm” or “neoplastic disease or disorder” refers to a primary or metastatic mass of tissue or cells, particularly bone cells, characterized by, inter alia, abnormal cell proliferation. The abnormal cell proliferation results in growth of these tissues or cells that exceeds and is uncoordinated with that of the normal tissues or cells and persists in the same excessive manner after the stimuli which evoked the change ceases or is removed. Neoplastic tissues or cells show a lack of structural organization and coordination relative to normal tissues or cells which usually results in a mass of tissues or cells which can be either benign or malignant. As would be apparent to one of ordinary skill in the art, the term “cancer” refers to a malignant neoplasm.

As used herein, the terms “treating” or “treatment” includes, but is not limited to, prophylactic treatment, pain palliation and alleviation of ongoing or intermittent symptoms, including remission or cure, occurring in a bone-associated pathological condition. For a primary or metastatic neoplastic bone disease or disorder this includes, but is not limited to, halting the growth of the neoplasm or cancer, killing the neoplasm or cancer, or reducing the size of the neoplasm or cancer. Halting the growth refers to halting any increase in the size or the number of or size of the neoplastic or cancer cells or to halting the division of the neoplasm or the cancer cells. Reducing the size refers to reducing the size of the neoplasm or the cancer or the number of or size of the neoplastic or cancer cells.

As used herein, the term “subject” refers to any recipient of tin-117m radiotherapy.

In one embodiment of the present invention there is provided a radiotherapeutic composition, comprising a high specific activity tin-117m source of radiotherapeutic atomic electrons; and a delivery vehicle effective to contain tin-117m therein. Further to this embodiment the radiotherapeutic composition may comprise a targeting moiety linked to the delivery vehicle. Examples of a targeting moiety are an antibody or ligand effective to target a cell characteristic of the bone-associated pathological condition.

In these embodiments the delivery vehicle may be a chelator. Examples of chelators are 1-hydroxyethylidine-1,1-diphosphonic acid (HEDP), diethylenetriaminepentaacetic acid (DTPA), methylene diphosphonate (MDP), pyrophosphate (PYP), ethylidenehydroxydisodium phosphonate (EHDP).

Alternatively, in these embodiments the delivery vehicle may be a nanosphere, a microsphere, a peptide, a fusion protein, a virus, or a bacterium. Also, the nanosphere or microsphere further may comprise a chelator chelated to the tin-117m. Examples of these chelators are as described supra. In all aspects of these embodiments the high specific activity is about 4 Ci/g to about 1,000 Ci/g. In addition, in these embodiments the radiotherapeutic electrons may be conversion electrons. Furthermore, the tin-117m is stannous tin-117m or stannic tin-117m.

In one aspect of these embodiments the radiotherapeutic composition may comprise a high specific activity stannous-117m chloride; a nanosphere or microsphere encapsulating the stannous-117m chloride; and a targeting moiety linked to the nanosphere or microsphere. In another aspect the radiotherapeutic composition may comprise a high specific activity stannic-117m chloride; a bifunctional chelator of stannic-117m chloride; and a targeting moiety linked to the bifunctional chelator. In both aspects the targeting moiety may be an antibody or ligand effective to target a cell characteristic of the bone-associated pathological condition.

In another embodiment of the present invention there is provided a method for treating a bone-associated pathological condition in a subject, comprising administering any of the radiotherapeutic compositions described supra to the subject where the radiotherapeutic atomic electrons emitted by the high specific activity tin-117m comprising the composition destroy cells upon delivery thereto thereby treating the bone-associated pathological condition. Further to this embodiment the method comprises imaging gamma emissions from the high specific activity tin-117m to monitor delivery of the radiotherapeutic composition to a site of the bone-associated pathological condition or to monitor efficacy of treatment or a combination thereof.

In both embodiments the the bone-associated pathophysiological condition is a neoplastic bone disease or disorder or a bone disease or disorder having an inflammatory component. Examples of a neoplastic bone disease are a primary or metastatic bone cancer. Examples of a bone disease or disorder having an inflammatory component is an osteoarthritis, rheumatoid arthritis, ankylosing spondylitis, or hemophilia.

The present invention provides compositions comprising high specific activity tin-117m as a source of radiotherapeutically useful atomic electrons, e.g., low energy, short range conversion electrons. In addition tin-117m provides gamma rays suitable for radioimaging. Preferably, tin-117 is irradiated or is activated in a reactor having a substantial flux of fast neutrons, i.e., those produced in a high flux fast reactor and/or in a high flux thermal reactor, for a period of time at high neutron energies effective to drive the reaction ¹¹⁷Sn (n, n′, γ) ^{117m}Sn. Alternatively, tin-116 may be irradiated or activated to produce the reaction ¹¹⁶Sn (n, γ) ^{117m}Sn.

Although the limiting specific activity of tin-117m is about 81,000 Ci/g or 3×10¹⁵ becquerels (3 PBq), such very high levels of specific activity currently are unattainable using even a high flux fast reactors. Prior art methods of tin-117m production can produce specific activities of 20 Ci/g. The present invention, however, provides tin-117m with a high specific activity of about 4 Ci/g to about 1000 Ci/g.

The high specific activity tin-117m isotope may be chelated to form a stannic tin-117m complex. Examples of chelators useful to carry tin-117m to a site of interest in a subject, i.e. a site comprising a primary or a metastatic bone neoplasm, may be, but not limited to, 1-hydroxyethylidine-1,1-diphosphonic acid (HEDP), diethylenetriaminepentaacetic acid (DTPA), methylene diphosphonate (MDP), pyrophosphate (PYP), ethylidenehydroxydisodium phosphonate (EHDP). As described herein, a suitable bifunctional chelant also may be linked to a targeting moiety.

Alternatively, the high specific activity tin-117m isotope may be encapsulated in nanospheres or microspheres. Also, as described herein the nanospheres or microspheres further may comprise a targeting moiety. Nanospheres and microspheres are well-known as delivery vehicles and the methods of preparation and loading procedures also are well-known and standard in the art. In addition the delivery vehicle may be a peptide, a fusion protein, a virus, or a bacterium which may further comprise a chelator effective for tin-117m. Methods of incorporating, encapsulating, linking or chelating a radioisotope to these delivery vehicles are standard and well-known in the art

The tin-117m isotope may be encapsulated as the stannous chloride, SnCl₂, or as the stannic tin-117m chelated complex described herein. The atomic electrons emitted from tin-117m are short range and passage of the emitted atomic electrons out of the nanosphere or microsphere may be attenuated by the composition of the nanospheres or microspheres itself. In the present invention, however, a sufficient amount of the high specific activity tin-117m isotope can be encapsulated to provide enough activity to remedy the attenuation effects of the nanospheres or microspheres. Thus, over time a radiotherapeutic amount of emitted atomic electrons will pass through the shell of the nanosphere or microsphere to treat a bone-associated pathophysiological condition.

Furthermore, the tin-117m chelated complex and nanospheres or microspheres encapsulating the tin-117m compound or complex may comprise a targeting moiety. Although tin-117m or chelated tin-117m gravitates to cortical bone tissue and does not exhibit adverse or toxic effects to normal healthy tissue, such as bone marrow, the addition of a targeting moiety provides a helper function. For example, a chelator may be a bifunctional chelator linked to a targeting moiety. Alternatively, the nanosphere or microsphere shell or corona or other delivery vehicle as described herein may comprise the targeting moiety linked thereto. Examples of targeting moieties are antibodies or ligands directed to appropriate receptors on cells characteristic of a bone-associated pathophysiological condition, e.g., but not limited to, neoplastic bone cells. Preferably, the antibodies or ligands are internalized by the targeted cells. The antibodies or ligands useful to selectively target such cells and the methods of linking are well known in the art.

Thus, the present invention provides methods of radiotherapy to treat bone-associated pathological conditions. For example, the radiotherapeutic methods provided herein may be used to treat primary or metastatic neoplastic bone diseases or disorders, e.g., bone cancers, such that neoplastic cell proliferation is arrested, stopped, blocked, or ceases to occur without affecting normal untransformed cell function, such as bone marrow function. It is well-known that primary cancers such as prostate, breast or lung cancers metastasize to the bone. In addition radiotherapy with high specific activity tin-117m may be used to treat a primary bone neoplasm, such as, but not limited, to osteosarcoma or Ewing's sarcoma. It is contemplated that tin-117m may be useful in the treatment of multiple myeloma.

Also, these radiotherapeutic methods may be useful to treat bone-associated diseases or disorders having an inflammatory component, including autoimmune and genetic diseases and disorders. Without being limiting an example of a bone-associated autoimmune disease may be arthritis, such as osteoarthritis, rheumatoid arthritis or ankylosing spondylitis. In addition and still without being limiting, an example of a bone-associated genetic disorder is hemophilia. It is well-established that these conditions may cause joint inflammation and/or other pathologies of the bone.

A subject in need of such treatment may receive radiotherapeutic atomic electrons from the high specific activity tin-117m source described herein. It is contemplated that such radiotherapy may result in a substantial therapeutic benefit toward the bone-associated pathological condition. In the case of a primary neoplastic bone disease or disorder, treatment may effect a material extension of the subject's life, including remission or a cure. As is known in the art, cure is defined, for example in the United States, as at least 5-year disease-free survival.

Dosage formulations of high specific activity tin-117m, for example, stannous chloride, SnCl₂ encapsulated within or stannic tin chelated within a nanosphere or microsphere or complexed to a chelate as stannic tin-117m, also may comprise conventional non-toxic, physiologically or pharmaceutically acceptable carriers or other vehicles suitable for the method of administration. Routes of administration are well-known and standard in the art and include, but are not limited to, direct injection or intravenous administration of the radiotherapeutic composition.

These high specific activity tin-117m compounds, compositions or sources or pharmaceutical compositions thereof may be administered independently one or more times to achieve, maintain or improve upon a radiotherapeutic effect derived therefrom or to augment a pharmacologic or therapeutic effect derived from other agents suitable to treat the primary or metastatic neoplastic disease or disorder. It is well within the skill of an artisan to determine dosage or whether a suitable dosage comprises a single administered dose or multiple administered doses of the tin-117m isotope. An appropriate dosage depends on the subject's health, the progression or remission or the at risk status for the bone-associated pathological condition, the route of administration, the formulation used, and the high specific activity of the tin-117m administered to the subject.

The present invention further provides methods of radioimaging by detecting the gamma ray emitted by tin-117m. A gamma ray of about 159 keV is present in 86% of emissions from tin-117m. Radioimaging of one or more bones of the subject receiving tin-117m radiotherapy is useful to locate the tin-117m isotope and/or to monitor the treatment, the progression or the remission of the bone-associated pathological condition. Methods of external gamma imaging, e.g., planar, Positron Emission Tomography (PET), Single Photon Emission Computed Tomography (SPECT) and bone scintigraphy are well-known and standard in the art.

One skilled in the art will readily appreciate that the present invention is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A radiotherapeutic composition, comprising:
a high specific activity tin-117m source of radiotherapeutic atomic electrons; and
a delivery vehicle effective to contain tin-117m therein.

2. The radiotherapeutic composition of claim 1, further comprising a targeting moiety linked to said delivery vehicle.

3. The radiotherapeutic composition of claim 2, wherein said targeting moiety is an antibody or ligand effective to target a cell characteristic of the bone-associated pathological condition.

4. The radiotherapeutic composition of claim 1, wherein said delivery vehicle is a chelator.

5. The radiotherapeutic composition of claim 4, wherein said chelator is 1-hydroxyethylidine-1,1-diphosphonic acid (HEDP), diethylenetriaminepentaacetic acid (DTPA), methylene diphosphonate (MDP), pyrophosphate (PYP), ethylidenehydroxydisodium phosphonate (EHDP).

6. The radiotherapeutic composition of claim 1, wherein said vehicle is a nanosphere, a microsphere, a peptide, a fusion protein, a virus, or a bacterium.

7. The radiotherapeutic composition of claim 6, further comprising a chelator chelated to said tin-117m.

8. The radiotherapeutic composition of claim 7, wherein said chelator is 1-hydroxyethylidine-1,1-diphosphonic acid (HEDP), diethylenetriaminepentaacetic acid (DTPA), methylene diphosphonate (MDP), pyrophosphate (PYP), ethylidenehydroxydisodium phosphonate (EHDP).

9. The radiotherapeutic composition of claim 1, wherein said high specific activity is about 4 Ci/g to about 1000 Ci/g.

10. The radiotherapeutic composition of claim 1, wherein said atomic electrons are conversion electrons.

11. The radiotherapeutic composition of claim 1, wherein said tin-117m is stannous tin-117m or stannic tin-117m.

12. A method for treating a bone-associated pathological condition in a subject, comprising:
administering the radiotherapeutic composition of claim ? to the subject wherein said radiotherapeutic atomic electrons emitted by the high specific activity tin-117m comprising said composition destroys cells characteristic of the bone-associated pathological condition upon delivery thereto thereby treating the pathophysiological bone condition.

13. The method of claim 12, further comprising:
imaging gamma emissions from said high specific activity tin-117m to monitor delivery of the radiotherapeutic composition to a site of the bone-associated pathological condition or to monitor efficacy of treatment or a combination thereof.

14. The method of claim 12, wherein the bone-associated pathophysiological condition is a neoplastic bone disease or disorder or a bone disease or disorder having an inflammatory component.

15. The method of claim 14, wherein said neoplastic bone disease is a primary or metastatic bone cancer.

16. The method of claim 14, wherein the bone disease or disorder having an inflammatory component is an osteoarthritis, rheumatoid arthritis, ankylosing spondylitis, or hemophilia.

17. A radiotherapeutic composition, comprising:
a high specific activity tin-117m source of radiotherapeutic atomic electrons; and
a nanosphere or microsphere encapsulating said tin-117m.

18. The radiotherapeutic composition of claim 17, further comprising a targeting moiety linked to said nanosphere or microsphere.

19. The radiotherapeutic composition of claim 18, wherein said targeting moiety is an antibody or ligand effective to target a cell characteristic of the bone-associated pathological condition.

20. The radiotherapeutic composition of claim 17, wherein said encapsulated tin-117m is stannous chloride.

21. The radiotherapeutic composition of claim 17, further comprising a chelator chelated to said tin-117m.

22. The radiotherapeutic composition of claim 21, wherein said chelated tin-117m is stannic tin-117m.

23. The radiotherapeutic composition of claim 22, wherein said chelator is 1-hydroxyethylidine-1,1-diphosphonic acid (HEDP), diethylenetriaminepentaacetic acid (DTPA), methylene diphosphonate (MDP), pyrophosphate (PYP), ethylidenehydroxydisodium phosphonate (EHDP).

24. The radiotherapeutic composition of claim 17, wherein said high specific activity is about 4 Ci/g to about 1000 Ci/g.

25. The radiotherapeutic composition of claim 17, wherein said atomic electrons are conversion electrons.

26. A method for treating a bone-associated pathological condition in a subject, comprising:
administering the radiotherapeutic composition of claim 17 to the subject wherein said radiotherapeutic atomic electrons emitted by the high specific activity tin-117m comprising said composition destroys cells characteristic of the bone-associated pathological condition upon delivery thereto thereby treating the pathophysiological bone condition.

27. The method of claim 26, further comprising:
imaging gamma emissions from said high specific activity tin-117m to monitor delivery of the radiotherapeutic composition to a site of the bone-associated pathological condition or to monitor efficacy of treatment or a combination thereof.

28. The method of claim 26, wherein the bone-associated pathophysiological condition is a neoplastic bone disease or disorder or a bone disease or disorder having an inflammatory component.

29. The method of claim 28, wherein said neoplastic bone disease is a primary or metastatic bone cancer.

30. The method of claim 28, wherein the bone disease or disorder having an inflammatory component is an osteoarthritis, rheumatoid arthritis, ankylosing spondylitis, or hemophilia.

31. A radiotherapeutic composition, comprising:
a high specific activity tin-117m source of radiotherapeutic atomic electrons; and
a chelator of tin-117m.

32. The radiotherapeutic composition of claim 31, further comprising a targeting moiety linked to said delivery vehicle.

33. The radiotherapeutic composition of claim 32, wherein said targeting moiety is an antibody or ligand effective to target a cell characteristic of the bone-associated pathological condition.

34. The radiotherapeutic composition of claim 31, wherein said chelator is 1-hydroxyethylidine-1,1-diphosphonic acid (HEDP), diethylenetriaminepentaacetic acid (DTPA), methylene diphosphonate (MDP), pyrophosphate (PYP), ethylidenehydroxydisodium phosphonate (EHDP).

35. The radiotherapeutic composition of claim 31, wherein said chelated tin-117m is stannic tin-117m.

36. The radiotherapeutic composition of claim 31, wherein said high specific activity is about 4 Ci/g to about 1000 Ci/g.

37. The radiotherapeutic composition of claim 31, wherein said atomic electrons are conversion electrons.

38. A method for treating a bone-associated pathological condition in a subject, comprising:
administering the radiotherapeutic composition of claim 31 to the subject wherein said radiotherapeutic atomic electrons emitted by the high specific activity tin-117m comprising said composition destroys cells characteristic of the bone-associated pathological condition upon delivery thereto thereby treating the pathophysiological bone condition.

39. The method of claim 38, further comprising:
imaging gamma emissions from said high specific activity tin-117m to monitor delivery of the radiotherapeutic composition to a site of the bone-associated pathological condition or to monitor efficacy of treatment or a combination thereof.

40. The method of claim 38, wherein the bone-associated pathophysiological condition is a neoplastic bone disease or disorder or a bone disease or disorder having an inflammatory component.

41. The method of claim 40, wherein said neoplastic bone disease or disorder is a primary or metastatic bone cancer.

42. The method of claim 40, wherein the bone disease or disorder having an inflammatory component is an osteoarthritis, rheumatoid arthritis, ankylosing spondylitis, or hemophilia.
